(19) United States
(12) Patent Application Publication
(10) Pub. No.: US 2011/0098817 A1
(43) Pub. Date: Apr. 28, 2011

(54) SACRO-ILIAC JOINT IMPLANT SYSTEM AND METHOD

(75) Inventors: Jason J. Eckhardt, Memphis, TN (US); Mark Dace, Collierville, TN (US); Clinton R. Jacob, Germantown, TN (US)

(73) Assignee: WARSAW ORTHOPEDIC, INC., Warsaw, IN (US)

(21) Appl. No.: 12/607,477

(22) Filed: Oct. 28, 2009

(51) Int. Cl. A61F 2/44

(52) U.S. Cl. 623/17.11

(57) ABSTRACT

An orthopedic implant includes at least one elongated body. The body defines an outer surface. The outer surface is configured to engage an articular surface of a sacro-iliac joint along a plane substantially parallel to the articular surface.

### TECHNICAL FIELD

The present disclosure generally relates to medical devices for the treatment of musculoskeletal disorders, and more particularly to an implant system and method for treating the sacro-iliac joint.

### BACKGROUND

The sacroiliac (SI) joint is a diarthrodial joint that joins the sacrum to the ilium bones of the pelvis. In the SI joint, the sacral surface has hyaline cartilage that moves against fibrocartilage of the iliac surface. The spinal column is configured so that the weight of an upper body rests on the SI joints at the juncture of the sacrum and ilia. Stress placed on the SI joints in an upright position of the body makes the lower back susceptible to injury.

Disorders of the SI joint can cause low back and radiating buttock and leg pain in patients suffering from degeneration and laxity of the SI joint. In some cases, the SI joint can undergo dehydration and destabilization, similar to other cartilaginous joints, which causes significant pain. The SI joint is also susceptible to trauma and degeneration, from fracture and instability. It is estimated that disorders of the SI joint are a source of pain for millions of people suffering from back and radicular symptoms.

Non-surgical treatments, such as medication, injection, mobilization, rehabilitation and exercise can be effective, however, may fail to relieve the symptoms associated with these disorders. Surgical treatment of these disorders include stabilization and/or arthrodesis. Stabilization can include the use of bone screws that are directly threaded into bone. Arthrodesis may include fusion devices to immobilize a joint. The present disclosure describes an improvement over these prior art technologies.

### SUMMARY OF THE INVENTION

Accordingly, an implant system and method is provided for treating the SI joint. It is contemplated that the system may include a tubular implant configured for disposal with the SI joint. It is further contemplated that the implant system and method may be employed for arthrodesis and/or arthroplasty treatment.

In one particular embodiment, in accordance with the principles of the present disclosure, an orthopedic implant is provided. The orthopedic implant includes at least one elongated body. The body defines an outer surface. The outer surface is configured to engage an articular surface of a sacro-iliac joint along a plane substantially parallel to the articular surface.

### BRIEF DESCRIPTION OF THE DRAWINGS

The present disclosure will become more readily apparent from the specific description accompanied by the following drawings, in which:

FIG. 1 is a perspective view of one particular embodiment of an implant system in accordance with the principles of the present disclosure;

FIG. 2 is a plan view of the implant system shown in FIG. 1 and a SI joint;

FIG. 3 is a plan view of the implant system and SI joint shown in FIG. 2;

FIG. 4 is a plan view of one embodiment of the implant system shown in FIG. 2 and a SI joint;

FIG. 5 is a cutaway plan view of one embodiment of the implant system and SI joint shown in FIG. 2;

FIG. 6 is a plan view of one embodiment of the implant system and SI joint shown in FIG. 2;

FIG. 7 is a side section view of one embodiment of an implant system in accordance with the principles of the present disclosure;

FIG. 8 is a plan view of the implant system shown in FIG. 7 and a SI joint;

FIG. 9 is a plan view of the implant system shown in FIG. 7 and a SI joint;

FIG. 10 is a plan view of the implant system shown in FIG. 7 and a SI joint;

FIG. 11 is a cutaway perspective view of one embodiment of the implant system;

FIG. 12 is a cutaway plan view of the implant system shown in FIG. 11 and a SI joint;

FIG. 13 is a cutaway perspective view of one embodiment of the implant system;

FIG. 14 is a plan view of one embodiment of an implant system in accordance with the principles of the present disclosure and a SI joint;

FIG. 15 is a perspective view of one embodiment of an implant system in accordance with the principles of the present disclosure;

FIG. 16 is a front view of the implant system shown in FIG. 15;

FIG. 17 is a plan view of the implant system shown in FIG. 15 and a SI joint;

FIG. 18 is a plan view of the implant system shown in FIG. 15 and a SI joint; and

FIG. 19 is a plan view of one embodiment of the implant system shown in FIG. 15 and a SI joint.

Like reference numerals indicate similar parts throughout the figures.

### DETAILED DESCRIPTION OF THE INVENTION

The exemplary embodiments of the implant system and methods of use disclosed are discussed in terms of medical devices for the treatment of musculoskeletal disorders and more particularly, in terms of an implant system and method for treating the SI joint. It is envisioned that the implant system and methods of use disclosed provide stability and maintains structural integrity while reducing stress on the SI joint. It is further envisioned that the present disclosure may be employed to treat musculoskeletal disorders including sacro-Iliac dysfunction or syndrome, dehydration, destabilization, laxity, fracture, tumor, spinal disorders and other orthopedic disorders. It is contemplated that the present disclosure may be employed with surgical treatments, including open surgery, percutaneous and minimally invasive procedures of such disorders, such as, for example, arthrodesis including fusion, arthroplasty to maintain motion, bone graft and implantable prosthetics. It is further contemplated that the present disclosure may be employed with other osteal and bone related applications, including those associated with diagnostics and therapeutics. The disclosed implant system and methods may be employed in a surgical treatment with a patient in a prone or supine position, employing a posterior, lateral or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

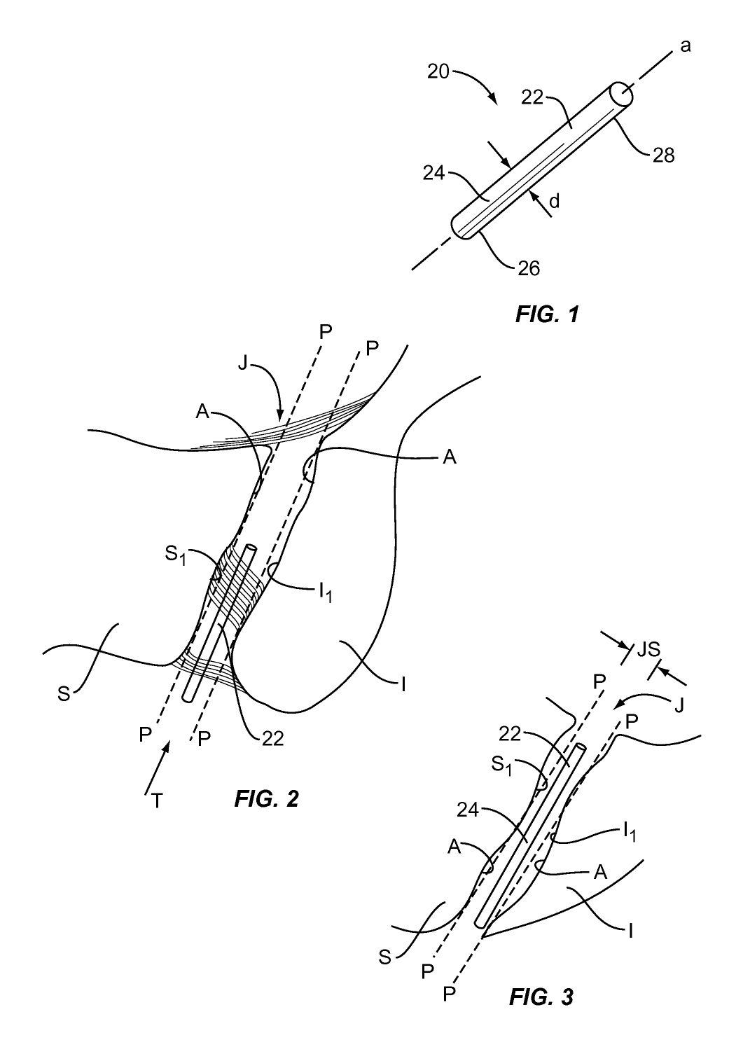

The following discussion includes a description of an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-3, there are illustrated components of the implant system in accordance with the principles of the present disclosure.

The components of the implant system are fabricated from materials suitable for medical applications, including metals, synthetic polymers, ceramics, bone, biocompatible materials and/or their composites, depending on the particular application and/or preference of a medical practitioner. For example, components of the implant system, such as, for example, a tubular body, an outer surface of the tubular body and/or an inner core and/or portions thereof, discussed below, can be fabricated from materials such as commercially pure titanium, titanium alloys, super-elastic titanium alloys, cobalt-chrome alloys, stainless steel alloys, thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof, rigid polymers including polyphenylene, polyamide, polyimide, polyetherimide, polyethylene, polyurethanes of any durometer, epoxy, silicone, bone material including autograft, allograft, xenograft or transgenic cortical and/or corticocancellous bone, and tissue growth or differentiation factors. Different components of the implant system may have alternative material composites to achieve various desired characteristics such as strength, rigidity, elasticity, compliance, biomechanical performance, durability and radiolucency or imaging preference.

It is envisioned that the components of the implant system can be manufactured via various methods. For example, the tubular body and the inner core can be manufactured and assembled via injection-molding, insert-molding, overmolding, compression molding, transfer molding, co-extrusion, pultrusion, dip-coating, spray-coating, powder-coating, porous-coating, milling from a solid stock material, and their combinations. One skilled in the art, however, will realize that such materials and fabrication methods suitable for assembly and manufacture, in accordance with the present disclosure, would be appropriate.

The implant system includes an orthopedic implant, such as, for example, a sacro-iliac implant 20, which is configured, for example, to treat S-I joint disorders including those caused by degeneration or trauma. It is contemplated that sacro-iliac implant 20 may be employed for arthrodesis and/or arthroplasty applications, as will be described.

Sacro-iliac implant 20 includes an elongated body 22 that defines an outer surface 24. Body 22 extends from a first end 26 to a second end 28 along a longitudinal axis a. Outer surface 24 is configured to engage an articular surface A of a sacro-iliac joint J along a plane P. Plane P is substantially parallel to articular surface A. It is contemplated that articular surface A may refer to a sacral surface S₁ of a sacrum S and/or an iliac surface I₁ of an ilium I. Body 22 may be solid or tubular, as will be described.

Body 22 has a cylindrical cross section and has a diameter d, according to the requirements of the particular application. It is envisioned that diameter d may be in a range of 2-25 millimeters (mm). It is contemplated that the cross-sectional geometry of body 22 may have various configurations, for example, round, oval, rectangular, polygonal, irregular, uniform, non-uniform, consistent or variable.

It is envisioned that body 22 can be variously configured and dimensioned with regard to size, shape, thickness, geometry and material. Body 22 may also be formed of one or a plurality of elements such as spaced apart portions, staggered patterns and mesh. It is envisioned that the particular geometry and material parameters of body 22 may be selected to modulate the flexibility or stiffness of sacro-iliac implant 20, such as those examples discussed herein. For example, body 22 can be configured to have varying ranges or degrees of flexibility or stiffness such as rigid, compliant, or reinforced. Depending on the flexibility or stiffness of body 22, the flexibility or stiffness of sacro-iliac implant 20 can be contoured according to the requirements of a particular application. It is contemplated that the ability to vary stiffness of sacro-iliac implant 20 provides restoration of kinematic function of joint J or promote fusion of the elements of joint J. It is envisioned that the components of sacro-iliac implant 20 may be monolithically formed, integrally connected or arranged with attaching elements.

In assembly, operation and use, the implant system including sacro-iliac implant 20 is employed with a surgical procedure for treatment of a sacro-iliac joint of a patient, as discussed herein. The implant system may also be employed with other surgical procedures. In particular, the implant system is employed with a surgical fusion procedure for treatment of a condition or injury, such as degeneration or fracture, of an affected sacro-iliac joint J, as shown in FIGS. 2 and 3. It is contemplated that the implant system is inserted with a sacro-iliac joint to provide a less invasive approach for treatment.

In use, to treat the affected section of sacro-iliac joint J, a medical practitioner obtains access to a surgical site including sacro-iliac joint J in any appropriate manner, such as through incision and retraction of tissues. It is envisioned that the implant system may be used in any existing surgical method or technique including open surgery, mini-open surgery, minimally invasive surgery and percutaneous surgical implantation, whereby sacro-iliac joint J is accessed through a mini-incision, or sleeve that provides a protected passageway to the area. Once access to the surgical site is obtained, the particular surgical procedure is performed for treating the sacro-iliac joint disorder. The implant system is then employed to augment the surgical treatment. The implant system can be delivered or implanted as a pre-assembled device or can be assembled in situ. The implant system may be completely or partially revised, removed or replaced in situ.

A trajectory T is defined for insertion of sacro-iliac implant 20 within sacro-iliac joint J. Trajectory T is determined by defining an orientation that is substantially parallel to plane P defined by articular surface A of sacro-iliac joint J. Implant 20 is inserted via the protected passageway along the defined trajectory T into sacro-iliac joint J. Sacro-iliac implant 20 is manipulated such that outer surface 24 of body 22 engages articular surface A substantially along plane P, according to the contour of articular surface A. Sacro-iliac implant 20 is disposed with sacro-iliac joint J for treating the sacro-iliac joint disorder. It is envisioned that body 22 may be inserted via a trajectory oriented from an anterior, posterior, superior or inferior direction.

It is envisioned that implant system can be used with various bone screws to enhance fixation. It is contemplated that the implant system and any screws and attachments may be coated with an osteoconductive material such as hydroxyapatite and/or osteoinductive agent such as a bone morphogenic protein for enhanced bony fixation to facilitate motion of the treated area. Sacro-iliac implant 20 can be made of radiolucent materials such as polymers. Radiomarkers may be included for identification under x-ray, fluoroscopy, CT or other imaging techniques.

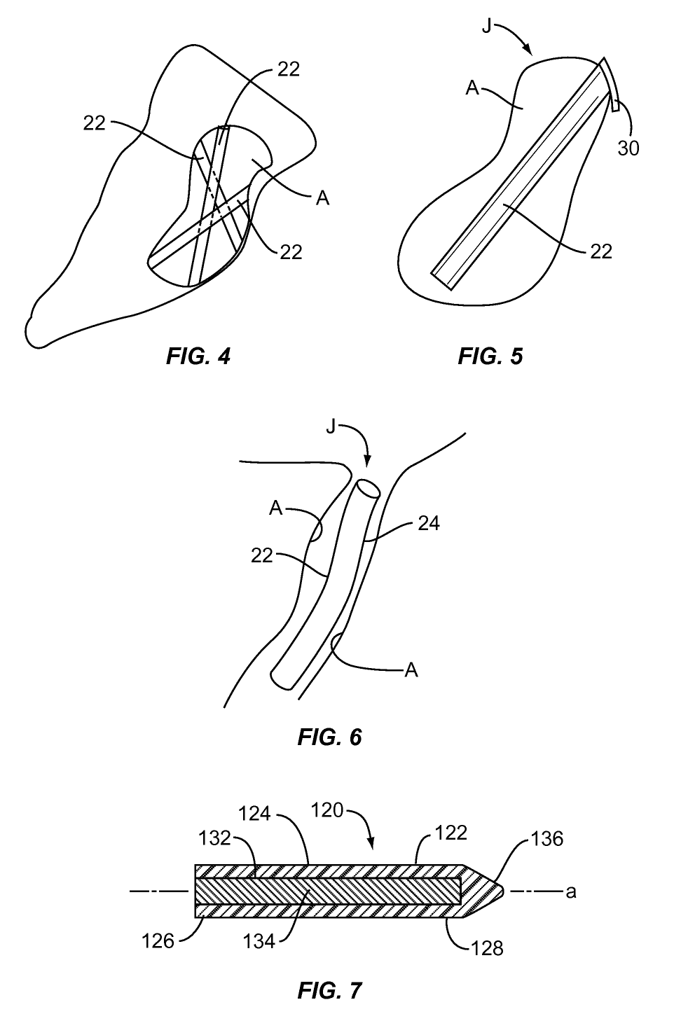

In one embodiment, as shown in FIG. 4, the implant system includes a plurality of cylindrical bodies 22, described above. It is contemplated that employing the plurality of bodies 22 can optimize the amount joint J can be spaced apart such that a joint space JS (FIG. 3) can be preselected. The plurality of bodies 22 can be inserted through the same or an alternate trajectory. The plurality of bodies 22 can be oriented in a side by side engagement, end to end engagement, end to side engagement, overlapping, spaced apart and/or staggered. It is envisioned that one or all of the plurality of bodies 22 may be inserted via a trajectory oriented from an anterior, posterior, superior or inferior directions. It is further envisioned that one or a plurality of bodies 22 may be used.

In one embodiment, as shown in FIG. 5, the implant system includes a cylindrical body 22, described above, having a locking part 30 configured for fixation with articular surface A. Locking part 30 engages a face or profile of articular surface A and can be employed with the implant system, for example, to facilitate fixation of body 22 with the joint surface during an arthrodesis treatment. Locking part 30 may engage an end or other surface of sacro-iliac joint J to fixate body 22. It is envisioned that locking part 30 may include one or a plurality of elements, may be variously connected body 22, or employ fastening elements such as, for example, clips, hooks, adhesives and/or flanges, as will be described below.

In one embodiment, as shown in FIG. 6, the implant system includes a cylindrical body 22, described above, having an undulating outer surface 24. The undulating configuration of outer surface 24 facilitates engagement of body 22 with the contour of sacro-iliac joint J. This engagement enhances compliance of sacro-iliac implant 20 with articular surfaces A.

Referring to FIGS. 7-10, in one embodiment similar to the implant system described above, a sacro-iliac implant 120 is configured, for example, to treat S-I joint disorders. It is contemplated that sacro-iliac implant 120 may be employed for arthrodesis and/or arthroplasty applications, as will be described.

Sacro-iliac implant 120 includes an elongated tubular body 122, similar to body 22 discussed above, which defines an outer surface 124. Body 122 extends from a first end 126 to a second end 128 along a longitudinal axis a.

Outer surface 124 is configured to engage an articular surface A of a sacro-iliac joint J along a plane P (shown in FIGS. 2 and 3). Plane P is substantially parallel to articular surface A. It is contemplated that articular surface A may refer to a sacral surface S1 of a sacrum S and/or an iliac surface I1 of an ilium I. Body 122 has a cylindrical cross section and has a diameter d. It is envisioned that diameter d may be in a range of 2-25 mm. It is envisioned that body 122 may be configured as a tubular spacer.

Body 122 defines an elongated cavity 132 configured for disposal of an inner core 134. It is contemplated that body 122 is configured as a hollow rod that can be over-molded or slip fit on to inner core 134. It is further contemplated that sacro-iliac implant 120 can provide dual stiffness to the implant system. For example, body 122 includes a first material, which is soft or flexible. Such flexible materials can include polyurethane and silicone based materials, foam, as well other flexible materials discussed herein. Inner core 134 is a rigid rod, which includes a rigid material, such as metals, rigid polymers and ceramics, as well other rigid materials discussed herein. It is envisioned that body 122 and inner core 134 may be formed from the same material, different materials or composites thereof. Body 122 may also be formed of semi-rigid and rigid materials, and inner core 134 may be formed of flexible or semi-rigid materials. Body 122 may be monolithically formed.

Depending on the flexibility or stiffness of body 122, the flexibility or stiffness of sacro-iliac implant 120 can be configured according to the requirements of a particular application. It is contemplated that the ability to vary stiffness of sacro-iliac implant 120 provides restoration of kinematic function of sacro-iliac joint J or promote fusion of the elements of sacro-iliac joint J.

Second end 128 has a tapered nose portion 136. Nose portion 136 facilitates impaction of sacro-iliac implant 120 within sacro-iliac joint J. It is contemplated that the configuration of nose portion 136 facilitates percutaneous access with sacro-iliac joint J.

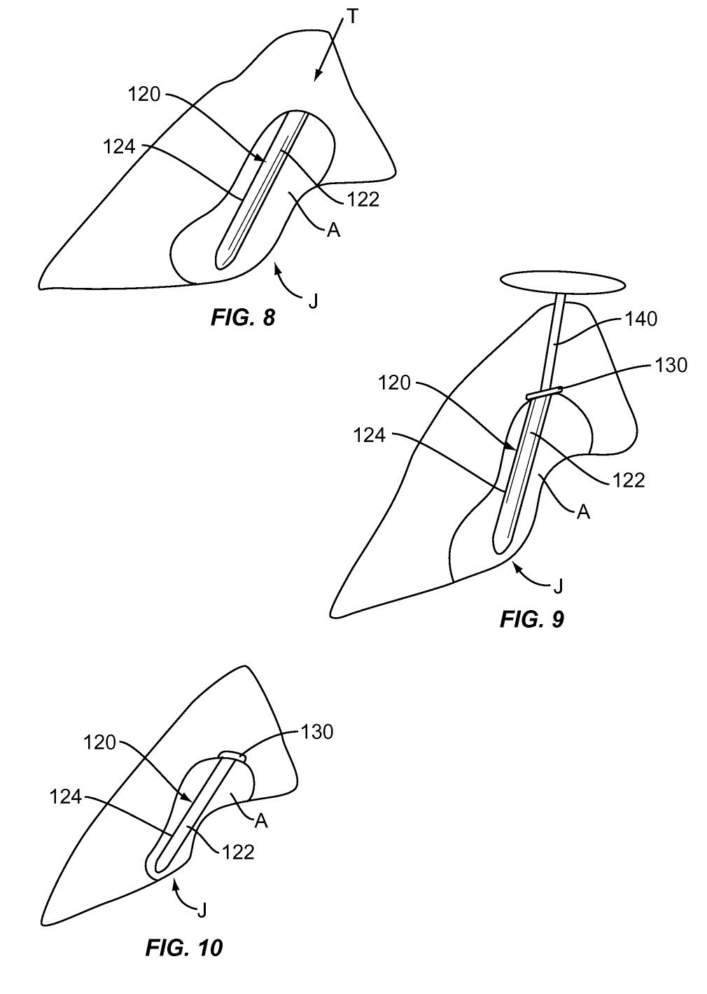

In assembly, operation and use, the implant system including sacro-iliac implant 120 is employed with a surgical procedure for treatment of a sacro-iliac joint of a patient and may be employed with other surgical procedures. Referring to FIGS. 8-10, the implant system is employed with a surgical fusion procedure for treatment of a condition or injury of sacro-iliac joint J, similar to that described with regard to FIGS. 2 and 3. In use, to treat the affected section of sacro-iliac joint J, a medical practitioner obtains access to a surgical site including sacro-iliac joint J.

A trajectory T (as described with regard to FIGS. 2 and 3) is defined for insertion of sacro-iliac implant 120 within sacro-iliac joint J. Implant 120 is inserted with an insertion tool 140 via a protected passageway along the defined trajectory T into sacro-iliac joint J. Sacro-iliac implant 120 is manipulated such that outer surface 124 of body 122 engages articular surface A along plane P. It is envisioned that insertion tool 140 may be threaded with an end portion of sacro-iliac implant 120 to facilitate disposal with sacro-iliac joint J, and subsequently unthreaded for removal of insertion tool 140 from sacro-iliac joint J. It is further envisioned that insertion tool 140 may be configured as an in-situ guidable instrument, and may include an endoscope camera tip for viewing insertion trajectory.

It is further envisioned that body 122 has a tubular and hollow configuration such that insertion tool 140 temporarily functions as an inner core for body 122 during insertion of sacro-iliac implant 120 with sacro-iliac joint J. In such a configuration, insertion tool 140 is removed such that body 122 is positioned for placement. Body 122 can be implanted and maintain rigidity or flatten under pressure.

Sacro-iliac implant 120 is disposed with sacro-iliac joint J for treating the sacro-iliac joint disorder. In one example, a procedure for implanting sacro-iliac implant 120 includes impaction of sacro-iliac implant 120 along plane P into joint space JS. The selected trajectory T and/or manipulation of body 122 after implantation can optimize positioning and/or placement of sacro-iliac implant 120. Manipulation can include pushing, pulling, rotation of sacro-iliac implant 120 about longitudinal axis a and/or rotation of sacro-iliac implant 120 about the joint axis once implanted. Fixation of sacro-iliac implant 120 with articular surface A and/or other portions of sacro-iliac joint J can be facilitated by the resistance provided by joint space JS and/or engagement with the outer articular structures. It is contemplated that the implant system including sacro-iliac implant 120 may be employed during arthoplasty.

Locking part 130 engages a face or profile of articular surface A to facilitate fixation of body 122 with sacro-iliac joint J during an arthrodesis treatment. Locking part 130 may include a flange, hook, hole, or void for primary fixation. It is contemplated that locking part 130 may include a secondary locking part for fixation such as screws and/or nails. It is further contemplated that locking part 130 can engage a face or profile of articular surface A, such as, for example, a crest portion of a surface of sacro-iliac joint J and/or an end component of joint space JS, for example to hook onto an end portion of ilium I. It is envisioned that in joint fusion applications of sacro-iliac implant 120, body 122 includes voids, cavities and/or openings for including bone promoting material, such as those described herein, which can be packed or otherwise disposed therein.

It is envisioned that body 122 may be inserted via a trajectory oriented from an anterior, posterior, superior or inferior direction. A portion or substantially all of outer surface 124 may be threaded for fixation of body 122 with articular surface A. Sacro-iliac implant 120 may be inserted parallel and/or in-line with plane P.

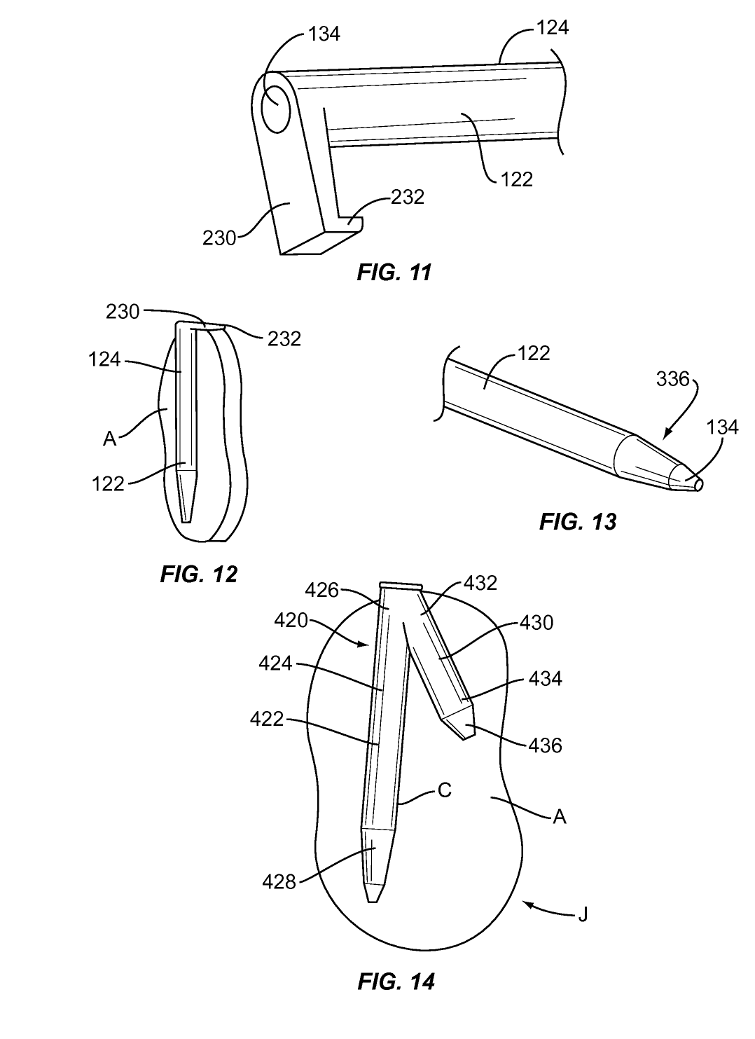

In one embodiment, as shown in FIGS. 11 and 12, the implant system includes cylindrical body 122, described above, having a locking part, such as, for example, a flange 230 extending radially from inner core 134 and outer surface 124. Flange 230 includes a retaining element 232 and is configured to engage articular surface A, for example, capturing the crest of ilium I or the sacrum S to fix body 122 with articular surface A.

In one embodiment, as shown in FIG. 13, the implant system includes cylindrical body 122, described above, having a chamfered nose portion 336. Inner core 134 is exposed at nose portion 336 and facilitates impaction of sacro-iliac implant 120 within sacro-iliac joint J.

In one embodiment, as shown in FIG. 14, the implant system has a sacro-iliac implant 420, which includes a first tubular body 422, similar to body 122 described above. Body 422 has an outer surface 424 and extends from a first end 426 to a second end 428. A second tubular body 430, similar to body 122 described above, is pivotally connected adjacent first end 426. Such pivotal connections can include a pin, a living hinge and/or a spring mechanism. It is contemplated that a pivotal component of body 422 and/or body 430, and/or a monolithic configuration thereof, is manipulated to plastically deform a continuous geometry of body 422 and/or body 430 into a selected angular shape. For example, a continuous body 422 and/or body 430 that is initially disposed with a 180 degree orientation can be manipulated to plastically deform to a 90 degree bend.

Second body 430 extends from a first end 432 to a second end 434 and includes a nose portion 436. Second body 430 may include an inner core, similar to those described above. First body 422 has a greater length relative to second body 430. During a surgical treatment of a disorder, similar to those described above, first body 422 is inserted along a channel C reamed along plane P (FIGS. 2 and 3) in articular surface A and parallel to sacro-iliac joint J. Second body 430 is pivoted into position relative to first body 422 for disposal and coverage of an alternate side of sacro-iliac joint J.

It is contemplated that first body 422 and second body 430 are employed to optimize the distance that sacro-iliac joint J can be spaced apart. This distance can be preselected. First body 422 and second body 430 can be inserted with sacro-iliac joint J through the same trajectory and pivoted from the other body. One or a plurality of pivoting bodies may be employed with first body 422. It is envisioned that a reamer or tapping device can be used to create a channel in articular surface A for receiving one or a plurality of tubular bodies. It is further envisioned that a guide wire system may be used to deliver the components of the implant system.

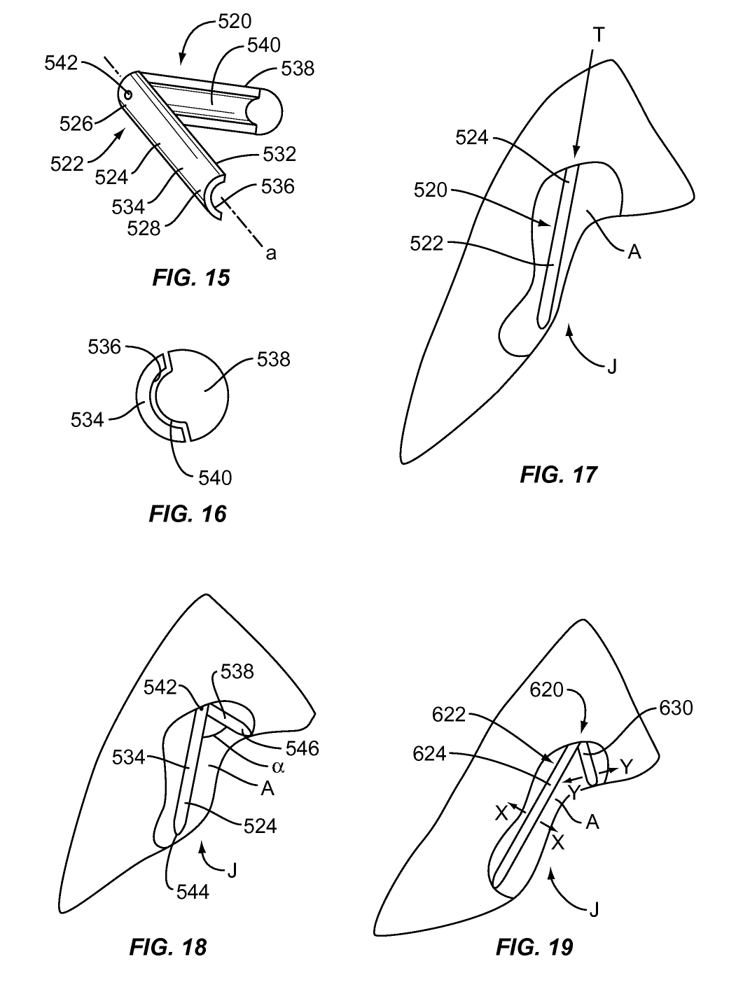

Referring to FIGS. 15-18, in one embodiment similar to the implant system described above, a sacro-iliac implant 520 is configured, for example, to treat S-I joint disorders. Sacro-iliac implant 520 includes an elongated tubular body 522, which defines an outer surface 524. Body 522 extends from a first end 526 to a second end 528 along a longitudinal axis a.

Outer surface 524 is configured to engage an articular surface A of a sacro-iliac joint J along a plane P, similar to that described above. Body 522 defines an elongated cavity 532. Body 522 includes a first elongated portion 534 defining an inner surface 536 and a second elongated portion 538 defining an inner surface 540 pivotally connected to first portion 534 adjacent first end 526. Inner surface 536 is concave and arcuate in a configuration for receiving inner surface 540, which has a convex configuration. Body 522 includes a pin 542 that connects first portion 534 with second portion 538 to facilitate pivotal connection therebetween.

Body 522 is disposable in a first orientation (FIG. 17) such that inner surface 536 and inner surface 540 are configured for mating engagement. Body 522 is disposable in a second orientation (FIG. 18) such that first portion 534 and second portion 538 are spaced apart for disposal with sacro-iliac joint J. First portion 534 has a tapered nose portion 544 and second portion 538 has a tapered nose portion 546.

In assembly, operation and use, the implant system including sacro-iliac implant 520 is employed with a surgical fusion procedure for treatment of a condition or injury of sacro-iliac joint J, similar to that described with regard to FIGS. 2 and 3. In use, to treat the affected section of sacro-iliac joint J, a medical practitioner obtains access to a surgical site including sacro-iliac joint J.

A trajectory T (as described with regard to FIGS. 2 and 3) is defined for insertion of sacro-iliac implant 520 within sacro-iliac joint J such that body 522 is disposed in the first orientation, as shown in FIG. 17. Sacro-iliac implant 520 is manipulated such that first portion 534 and second portion 538 are caused to separate in a lateral pivoting motion and space apart to the second orientation, as shown in FIG. 18.

In the second orientation, first portion 534 is disposed at an angle α from second portion 538. It is contemplated that angle α is disposed at a preselected angle α, which may be in a range of approximately 180 degrees to 90 degrees. It is further contemplated that angle α may be in a range of approximately 0-180 degrees. Outer surface 524 of body 522 engages articular surface A along plane P. Sacro-iliac implant 520 is disposed with sacro-iliac joint J for treating the sacro-iliac joint disorder. Manipulation of first portion 534 and second portion 538 to a spaced apart orientation, disposed at angle α, facilitates coverage of substantially the entire sacro-iliac joint J and spacing apart of the articular surfaces A.

Body 522 may include a locking part for fixing sacro-iliac implant 520 with articular surface A, similar to those described above. It is envisioned that body 522 may be inserted via a trajectory oriented from an anterior, posterior, superior or inferior direction.

In one embodiment, the implant system may be employed during an arthroplasty procedure. For example, as shown in FIG. 19, a sacro-iliac implant 620, similar to that described with regard to FIG. 14, is implanted with sacro-iliac joint J. Body 622 has an outer surface 624 and a second tubular body 630 pivots therefrom. Body 622 is configured for movable engagement with articular surface A in the direction of arrows X and body 630 is configured for movable engagement with articular surface A in the direction of arrows Y. Bodies 622, 630 roll along articular surface A allowing relative motion of the articular surfaces A of the sacrum and ilium of sacro-iliac joint J. The outer surfaces of bodies 622, 630 may be compressible.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A sacro-iliac implant comprising:
at least one elongated body defining an outer surface configured to engage an articular surface of a sacro-iliac joint along a plane substantially parallel to the articular surface.

2. A sacro-iliac implant according to claim 1, wherein the body is tubular and defines a cavity configured for disposal of an inner core.

3. A sacro-iliac implant according to claim 2, wherein the tubular body includes a first material and the inner core includes a second material different from the first material.

4. A sacro-iliac implant according to claim 2, wherein the tubular body is formed of a flexible material and the inner core is formed of a rigid material.

5. A sacro-iliac implant according to claim 2, wherein the tubular body extends from a first end to a second end, the second end being tapered.

6. A sacro-iliac implant according to claim 5, wherein the tapered second end includes a chamfered nose portion.

7. A sacro-iliac implant according to claim 1, wherein the body extends from a first end to a second end, the first end including a locking part configured for fixation with the articular surface or adjacent bone.

8. A sacro-iliac implant according to claim 7, wherein the locking part is a flange extending radially from the outer surface.

9. A sacro-iliac implant according to claim 1, wherein the at least one body includes a plurality of tubular bodies.

10. A sacro-iliac implant according to claim 1, wherein the at least one body includes a first tubular body extending from a first end to a second end and a second tubular body, the second tubular body being pivotally connected adjacent the first end of the first tubular body.

11. A sacro-iliac implant according to claim 2, wherein the tubular body extends from a first end to a second end and includes a first elongated portion defining an inner surface and a second elongated portion defining an inner surface pivotally connected to the first portion adjacent the first end.

12. A sacro-iliac implant according to claim 11, wherein the tubular body is disposable in a first orientation such that the inner surface of the first elongated portion and the inner surface of the second elongated portion are configured for mating engagement, and the tubular body is disposable in a second orientation such that the first elongated portion and the second elongated portion are spaced apart.

13. A sacro-iliac implant according to claim 1, wherein the outer surface is configured for movable engagement with the articular surface.

14. An orthopedic implant comprising:
an elongated tubular body extending from a first end to a second end having a tapered nose portion and defining an elongated cavity, the body including a flexible outer surface being circumferentially disposed about the body; and
an elongated rigid core disposed within the elongated cavity,
the outer surface being configured to engage an articular surface of a joint along a plane substantially parallel to a plane defined by the articular surface.

15. An orthopedic implant according to claim 14, wherein the tubular body extends from a first end to a second end, the first end including a locking part configured for fixation with the articular surface.

16. An orthopedic implant according to claim 15, wherein the locking part is a flange extending radially from the outer surface.

17. An orthopedic implant according to claim 14, further comprising a cylinder pivotally connected to the first end of the first tubular body.

18. An orthopedic implant according to claim 14, wherein the outer surface is configured for movable engagement with the articular surface.

19. A method for treating a sacro-iliac joint, the method comprising the steps of:
providing a sacro-iliac implant, the implant including at least one elongated tubular body defining an outer surface;
defining a trajectory for insertion of the implant within a sacro-iliac joint such that the trajectory is oriented substantially parallel to a plane defined by an articular surface of the sacro-iliac joint; and
inserting the implant via the defined trajectory into the sacro-iliac joint such that the outer surface of the body engages the articular surface along the plane defined by the articular surface.

20. A method for treating a sacro-iliac joint according to claim 19, wherein the outer surface of the body is disposed for movable engagement with the articular surface.
